(19) United States
(12) Patent Application Publication
(10) Pub. No.: US 2025/0288267 A1
(43) Pub. Date: Sep. 18, 2025

(54) ARTIFICIAL INTELLIGENCE SYSTEM, METHOD AND COMPUTER-ACCESSIBLE MEDIUM FOR MAMMOGRAPHY

(71) Applicant: NEW YORK UNIVERSITY, New York, NY (US)

(72) Inventors: Jungkyu PARK, New York, NY (US); Jan WITOWSKI, New York, NY (US); Krzysztof J. GERAS, Hoboken, NY (US)

(21) Appl. No.: 19/222,865

(22) Filed: May 29, 2025

Related U.S. Application Data
(63) Continuation of application No. PCT/US2023/081550, filed on Nov. 29, 2023.
(60) Provisional application No. 63/428,594, filed on Nov. 29, 2022.

(51) Int. Cl. A61B 6/50; A61B 6/02; G06T 7/00; G06T 7/10; G06T 15/00; G06V 10/25

(52) U.S. Cl. CPC A61B 6/502; A61B 6/025; G06T 7/0012; G06T 7/10; G06T 15/00; G06V 10/25; G06T 2207/20084; G06T 2207/30068; G06T 2207/30096

(57) ABSTRACT

Exemplary artificial intelligence (AI) systems, methods and computer-accessible medium can be provided for detecting a breast cancer using mammography. For example, using at least one computer processor, it is possible to receive a mammography image, apply a neural network employing, e.g., a You Only Look Once X (YOLOX) architecture to predict one or more locations and probabilities of lesions. Further, with such exemplary systems, methods and computer-accessible medium, it is possible to make an overall image-level prediction for the received mammography image so as to provide a particular breast cancer prediction. It is also possible to generate breast-level predictions by averaging all predictions from FFDM, C-View, DBT modalities for each breast. For example, when used in clinical decision support, the exemplary model can reduce radiologist workload by, e.g., about 45% and unnecessary recalls by, e.g., about 32.4%, without missing malignancies.

### CROSS REFERENCE TO RELATED APPLICATION(S)

This application relates to and claims the benefit of priority from U.S. Provisional Patent Application No. 63/428,594, filed on Nov. 29, 2022, the entire disclosure of which is incorporated herein by reference in its entirety.

### STATEMENT REGARDING FEDERALLY FUNDED RESEARCH

This invention was made with government support under Grant No. P41-EB017183 awarded by the National Institute of Health. Thus, the government has rights to the invention.

### FIELD OF THE DISCLOSURE

The present disclosure relates to mammography, and more specifically to exemplary systems, methods, and computer accessible medium for facilitating mammography, and detecting malignancies while minimizing false positives.

### BACKGROUND INFORMATION

Breast cancer is the leading cause of cancer-related deaths among women worldwide (see Sung et al., 2021). The lifetime risk of breast cancer for women is around 13% (see Howlader et al., 2020). Screening mammography has been shown to reduce the mortality rate for breast cancer (see Kopans, 2002; Duffy et al., 2002), but it is associated with a high rate of false-positive recalls and biopsies (see Kopans, 2015). In the United States, 39 million mammography exams are performed annually (see U.S. Food and Drug Administration, 2021). Out of the patients who undergo screening mammography, 10% are recalled (see Kopans, 2015) for additional imaging such as diagnostic mammograms, breast ultrasounds, and dynamic contrast-enhanced MRI. Out of the screened patients, 1-2% undergo a breast biopsy (see Kopans, 2015) which amounts to over 1.5 million women every year in the U.S. (see E1—more et al., 2015; Silverstein, 2009; Silverstein et al., 2009). Only 20-40% of biopsies yield cancer (see Kopans, 2015). The cost of diagnostic mammograms, breast ultrasounds, and biopsies are estimated to be $3.05 billion, $0.92 billion, and $3.07 billion annually (see Vlahiotis et al., 2018). This suggests that false-positive recalls cost $3.65 billion-$3.89 billion annually (excluding the cost of MRI exams), and false-positive biopsies cost $1.84 billion-$2.46 billion annually. In addition, biopsies are associated with pain and emotional distress for patients (see Hemmer et al., 2008; Maxwell et al., 2000), and can decrease the short-term quality of life (see Humphrey et al., 2014). The present artificial intelligence (AI) system to classify FFDM-DBT combo exams addresses this necessity to reduce the costs and harms related to false-positive findings of screening mammograms.

Digital breast tomosynthesis (DBT), a 3D imaging modality for breast cancer imaging, is poised to take over traditional 2D mammography. Although screening based on 3D mammography is more accurate than on 2D mammography, its interpretation time, of about 70 slices per volume, is almost doubled (see Aase et al., 2019). Thus, AI models with good performance in classifying mammography images for breast cancer can save radiologists a substantial amount of time.

Thus, it may be beneficial to provide an exemplary system, method, and computer accessible medium for detecting malignancies while minimizing false positives, which can overcome at least some of the deficiencies described herein above.

### SUMMARY OF EXEMPLARY EMBODIMENTS

The following is intended to be a brief summary of the exemplary embodiments of the present disclosure, and is not intended to limit the scope of the exemplary embodiments.

According to exemplary embodiments of the present disclosure, digital breast tomosynthesis (DBT) imaging techniques, procedures, systems, methods and computer-accessible medium, e.g., in mammography, can be provided. False positive recalls are still a subject of concern in the breast cancer screening setting, although it is more accurate than full-field digital mammography.

In certain exemplary embodiments of the present disclosure, an artificial intelligence (AI) systems, methods and computer-accessible medium can be provided which can use the screening mammography examination in a medical facility (e.g., at NYU Langone Health) which can save, e.g., about 32.39% of unnecessary recalls and potentially reduce radiologist workload by about 45% while missing no malignancies. For example, the exemplary AI system, method and computer-accessible medium can include and/or utilize deep neural networks trained on both breast-level labels and a limited amount of pixel-level segmentation labels. The exemplary AI system, method and computer-accessible medium can also indicate and/or highlight the location of suspicious findings on 2D and 3D mammography images for AI decision support.

To that end, according to certain exemplary embodiments of the present disclosure, artificial intelligence (AI) systems, methods, and computer-accessible medium can be provided to receive a mammography image, apply a neural network employing a You Only Look Once X (YOLOX) architecture to predict one or more locations and probabilities of lesions, aggregate one or more hidden representation corresponding to the resulting at least one bounding-box prediction, and generate an overall image-level prediction for the received mammography image so as to provide a particular prediction of the breast cancer.

For example, the mammography image can be or include a digital breast tomosynthesis (DBT) 3D image, a full-field digital mammography (FFDM) image, and/or a C-View image. When the mammography image is a digital breast tomosynthesis (DBT) 3D image, a neural network of the AI systems, methods, and/or computer-accessible medium can be configured to performed a maximum intensity projection along a depth axis to match dimensions of a corresponding C-View image associated with the mammography image.

In certain exemplary embodiments of the present disclosure, the AI systems, methods, and/or computer-accessible medium can make and/or generate a separate prediction on each 2D slice in the DBT 3D image, and the at least one computer processor can generate a final prediction for the DBT 3D image by aggregating the predictions for individual 2D slices.

For example, each of the at least one bounding box predictions can be defined by a plurality of segmentation components defining the contours of a predicted lesion. Further, the YOLOX architecture can be or include a YOLOX-L architecture or a YOLOX-X architecture.

The at least one computer processor can be configured to utilize the mammography image to produce an image-level probability of malignancy using breast-level labels extracted from bounding-box labels. It may also be configured to generate at least one bounding-box prediction of one or more locations and probabilities of lesions based on the mammography image and wherein the at least one image-wise prediction is generated based on the at least one bounding-box prediction.

Further, exemplary AI systems, methods and computer-accessible medium according to the exemplary embodiments of the present disclosure can be provided which can utilize the screening mammography exams which can save a large number of unnecessary recalls and potentially reduce radiologist workload by, e.g., 45% while missing no malignancies. For example, the exemplary systems, methods and computer-accessible medium can comprise at least one computer processor which can be used to train deep neural network (s) on, e.g., breast-level labels and a limited amount of pixel-level segmentation labels. The exemplary systems, methods and computer-accessible medium can also highlight the location of suspicious findings on 2D and 3D mammography images for AI decision support.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a flow diagram of an exemplary method for filtering of a test subset of screening mammography dataset according to an exemplary embodiment of the present disclosure;

FIG. 2 is a set of exemplary visualizations including exemplary model predictions of an anatomical structure according to an exemplary embodiment of the present disclosure;

FIG. 3 is a block diagram of an exemplary embodiment of a system according to the present disclosure; and

FIG. 4 is a flow chart of a method according to an exemplary embodiment of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXAMPLE EMBODIMENTS

The following description of exemplary embodiments provides non-limiting representative examples referencing numerals to particularly describe features and teachings of different exemplary aspects and exemplary embodiments of the present disclosure. The exemplary embodiments described should be recognized as capable of implementation separately, or in combination, with other exemplary embodiments from the description of the exemplary embodiments. A person of ordinary skill in the art reviewing the description of the exemplary embodiments should be able to learn and understand the different described aspects of the present disclosure. The description of the exemplary embodiments should facilitate understanding of the exemplary embodiments of the present disclosure to such an extent that other implementations, not specifically covered but within the knowledge of a person of skill in the art having read the description of embodiments, would be understood to be consistent with an application of the exemplary embodiments of the present disclosure.

### Exemplary Data Set Construction

As explained herein, the exemplary systems, method and computer-accessible medium according to the exemplary embodiment of the present disclosure can utilize, e.g., all of the three input modalities used in screening mammography: Full-Field Digital Mammography (FFDM), C-View, and Digital Breast Tomosynthesis (DBT). The three modalities have various benefits, and image the tissue differently. DBT is a 3D input modality that can include multiple slices which show structures appearing at corresponding depths. This reduces tissue overlap between nearby structures and thus increases lesion conspicuity.

DBT and C-View image quality can be compromised by artifacts created around calcification (see Horvat et al., 2019). In addition, 2D images such as FFDM and C-View are more useful than DBT for quickly scanning for clusters of calcifications. Since no single input modality is strictly superior, the modeling system disclosed herein can utilize all of the available input modalities. Concretely, the exemplary system, method, and computer accessible medium according to the exemplary embodiment of the present disclosure can, e.g., first compute the predictions for the three input modalities separately from models trained on each modality and average the predictions for each breast. This can lead to improved performances as shown in Table 3.

An exemplary screening mammography dataset includes 549,415 exams performed between 2010.01.04 and 2020.8.31 at NYU Langone Health. Similarly, an exemplary diagnostic mammography dataset consists of 111,455 exams performed between 2011.06.25 and 2021.07.09 at NYU Langone Health. This excludes all patients who had a screening exam in April-August of 2020 from training and validating the AI models to simulate a similar setting as deploying the AI model to the hospital. All other diagnostic exams which appeared on or after 2020.01.01 may be excluded. As a result, 153,029 exams can be excluded from the exemplary screening mammography dataset, and 44,602 exams can be excluded from the diagnostic mammography dataset. The remaining data can be randomly split with a 9:1 ratio of patients between the training and validation set as shown in Table 1 and Table 2. Finally, the AI model of exemplary system, method, and computer accessible medium can use 21,458 screening mammography exams acquired between January and March of 2020 to evaluate the AI model's performances.

TABLE 1
Summary of the number of screening mammography exams
in the dataset. The excluded exams (e.g., exams which
belong to patients who had a screening exam in April-
August of 2020) are not shown in this table because
they postdate the test set and therefore are not used
in training or evaluating the exemplary AI model.

| | DBT + FFDM | exams with FFDM | |
|---|---|---|---|
| | Combo exams | images only | Total |
| Training | 173,681 | 163,567 | 337,248 |
| Validation | 19,254 | 18,426 | 37,680 |
| Test (2020 Q1) | 21,458 | 0 | 21,458 |
| Total | 214,393 | 181,993 | 396,386 |

TABLE 2
Summary of the number of diagnostic mammography exams in
the dataset. Excluded exams (e.g., exams which belong to
patients who had a screening exam in April-August of 2020,
as well as diagnostic exams on or after Jan. 1st, 2020)
are not shown in this table because they are not used in
either training or evaluating the exemplary AI model.

| | DBT + FFDM | exams with FFDM | |
|---|---|---|---|
| | Combo exams | images only | Total |
| Training | 42,942 | 57,414 | 100,356 |
| Validation | 4,680 | 6,419 | 11,099 |
| Total | 47,622 | 63,833 | 111,455 |

### Exemplary Ground Truth Classification Labels

Using pathology reports associated with patients in the dataset, a ground truth label for each breast in the dataset is established. Exemplary categories for a ground truth label can be distinguished, as follows: malignant, benign (biopsy yielding benign findings), and negative (not biopsied). This exemplary label can be collected separately for each breast. In the exemplary screening mammography dataset, there are 2,754 breasts with pathology-proven breast cancer (2370, 244, and 140 breasts in training, validation and test sets, respectively), and 10,953 breasts (9,354, 1,010, 589 in training, validation and test sets, respectively) with a benign label (biopsies yielding benign results). Because one breast can be associated with multiple pathology findings of different types, malignant and benign labels are not mutually exclusive. Remaining breasts were negative. In the diagnostic mammography dataset, there are 5,441 breasts with pathology-proven breast cancer (4,887 and 554 breasts in training and validation sets, respectively) and 16,464 breasts (14,838 and 1,626 in training and validation sets, respectively) with benign findings.

### Exemplary Pixel-Level Lesion Labels

Radiologists may manually generate pixel-level annotations of lesions in the dataset. They can be asked to perform segmentation of lesions (contouring lesions of interest) visible on mammography images. These annotations have been acquired on a part of the full dataset. In the exemplary screening mammography dataset, there are 2459, 250, and 0 images with malignant annotations and 6221, 722, 0 images with benign annotations in the training, validation and test set, respectively. In the diagnostic mammography dataset, there are 239 and 21 images with malignant annotations and 105 and 15 images with benign annotations in the training and validation set, respectively.

For FFDM images in FFDM-only exams, a custom program according to exemplary embodiments can be used to acquire the segmentations. For DBT images, the segmentations for DBT images can be acquired using ITK-SNAP (see Yushkevich et al. 2006). To use 3D segmentations made on DBT images in 2D images, maximum intensity projection of 3D segmentations is first performed along the depth axis to match the C-View image dimensions. Then, these projected 2D segmentations can be resized to match the original dimensions of FFDM images.

### Exemplary Bounding-Box Labels

To train and evaluate object detection models, the AI system and method according to exemplary embodiments of the present disclosure can extract bounding-box labels from the pixel-level lesion labels. This can be performed by the exemplary AI system and method by defining a best-fit rectangle (aka bounding box, region of interest) for each connected component of the segmentation. In order to avoid drawing too many irrelevant bounding boxes, as a result of potential noise in segmentations, the AI system of exemplary embodiments can refine pixel-level annotations with morphological operations such as opening and closing to remove any artifacts.

### Exemplary Test Set Filtering Procedure

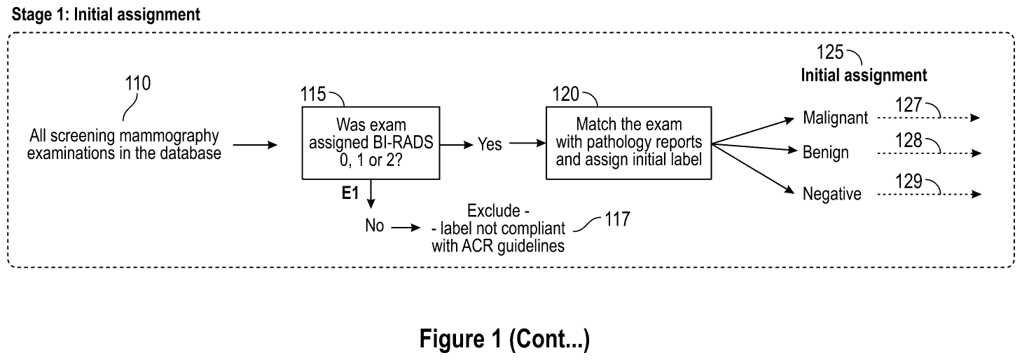

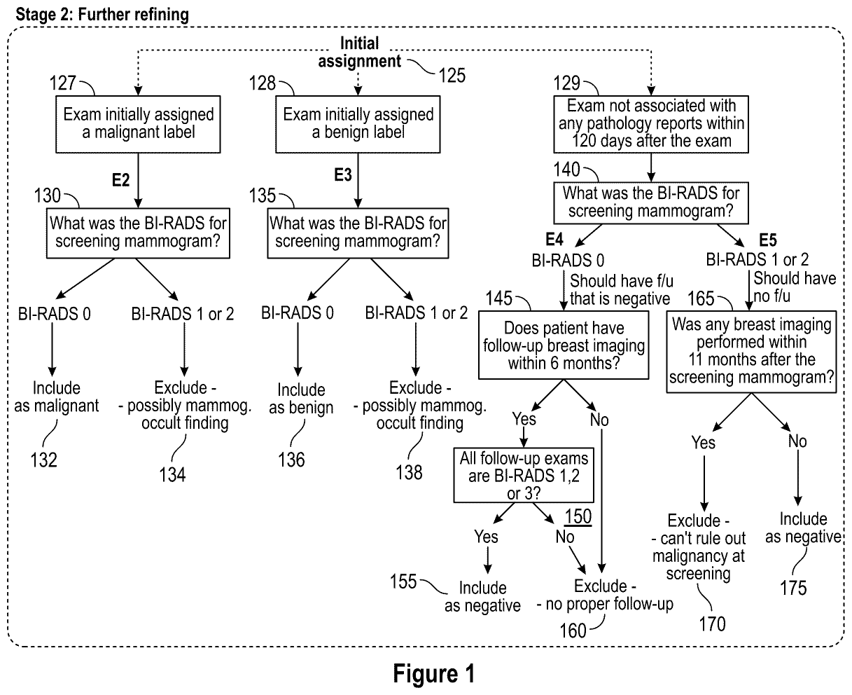

As a result of an exemplary test-set filtering procedure according to an exemplary embodiment of the present disclosure, as shown in a flow diagram of FIG. 1, 19,684 exams are selected in the test set. In particular, FIG. 1 illustrates a number of stages, which are as follows:

Stage 1: radiology reports and pathology reports are collected for all screening mammography examinations in the dataset at 110. Radiology reports can be used to collect BI-RADS category assigned to each exam at 115, and it is possible to use pathology reports to find information about diagnosis associated with the examination. E1: According to American College of Radiology guidelines, a screening exam should be assigned BI-RADS 0, 1 or 2. At this stage, all exams with other labels can be excluded at 117, because they may not properly represent screening mammography. For all remaining screening exams, they can be matched with breast pathology reports at 120, and use the diagnosis information to make an initial assignment 125 and classify them as malignant 127, benign 128, or negative 129. Exam is considered “negative” if it is not associated with any pathology report, and “benign” when there is a pathology report matching (e.g. biopsy), but there was no breast cancer found.

Stage 2: it is possible to perform exam filtering based on the initial assignment 125. E2: If an exam was assigned a malignant label 127, it is expected that screening mammography was given BI-RADS 0, and not BI-RADS 1 or 2 at 130. All BI-RADS 1/2 screening exams have been excluded at 134, as they could have been possibly mammographically occult and all BI-RADS 0 may be included as malignant at 132. E3: If an exam was assigned a benign label 128, it is also expected to be BI-RADS 0 at 135. For example, all BI-RADS 1/2 exams can be excluded at 138, while all BI-RADS 0 exams can be included at 136. If an exam was assigned a negative label 129, it is important to confirm that it is truly negative at 140. E4: If a mammogram with negative label was given BI-RADS 0, it is likely that there were suspicious findings which were later deemed to be benign or not found at all. To confirm this, at 145, all such cases should have at least one follow-up exam within 6 months. Furthermore, at 150, all exams in the next 6 months must be BI-RADS 1, 2 or 3. If this is the case, then at 155, it can be included as negative. If this is not the case, then it should be excluded at 160. E5: If a patient had a mammogram with negative label and BI-RADS 1 or 2, she should not have any breast imaging done before the next screening exam, which can be expected in 11-13 months. At 165, if that patient had any breast imaging performed within 11 months after the negative mammogram, this exam can be excluded at 170, otherwise the negative result may be included at 175. This rule can exclude some interval cancer patients.

Previously, an object detection system was developed to find pathology-proven lesions in DBT (see Park et al., 2021). EfficientDet (Tan et al., 2020) architecture was utilized to predict lesions on each 2D slice and combined the predictions for each 3D image. The exemplary AI model won the first and second phases of the SPIE-AAPM-NCI DAIR Digital Breast Tomosynthesis Lesion Detection (DBTex) Challenge (see Park et al. 2021).

### Exemplary Methods and Systems of Exemplary Embodiments

Specifically, the exemplary AI system and methods according to various exemplary embodiments of the present disclosure disclosed herein can include deep neural networks trained on both breast-level labels and a particular amount of bounding-box labels. While bounding-box labels are only available to a small subset of exams, it provides richer and more precise training signals to the model and leads to increased performances.

You only look once X (YOLOX) (see Ge et al., 2021) architecture can be utilized to predict the location of lesions in mammography images. These exemplary predictions can be made separately on each 2D image, specifically on each view in FFDM images and on each slice in DBT images. To generate a final prediction for a whole DBT image, the AI system according to exemplary embodiments, can aggregate the predictions for individual 2D slices.

The exemplary system and method according to the exemplary embodiments of the present disclosure can also highlight the location of suspicious findings on 2D and 3D mammography images for AI decision support. Both saliency maps and bounding-box predictions are available.

### Exemplary Image Loading and Augmentation

For example, mammography images can be loaded by cropping a window of pre-determined size. The size of the cropping window can be 2866×1814 for FFDM and 2166×1339 for DBT and C-View images to handle the respective original image resolutions. These cropped images can then be resized to 1536×1024, 1664×1152 or 1920×1280 for model processing as they may show the best performances in a hyperparameter search. These sizes are only a few selected examples as the exemplary AI system can be capable of processing any image sizes which are multiples of 128, as well as multiples of others.

Exemplary Training In exemplary embodiments of the present disclosure, the cropping window can be placed in all possible locations on the background-removed mammograms. The details on the background-removal process are in the earlier data report (see Wu et al., 2019). An exemplary system in turn can apply random horizontal and/or vertical flips as well as affine transformation to both image and segmentation. The flip probabilities are 0.5, and the affine transformation parameters are as follows:
1. rotation: ±15 degrees
2. translation: ±10% in both width and height directions
3. scale: 80%-160%
4. shear: ±25 degrees

The bounding boxes can be extracted again from such augmented segmentation to ensure the tightest fit to the lesion.

Exemplary Validation The cropping window can be placed in the location determined to be optimal. (See Wu et al., 2019).

### Exemplary Test

At test time, the exemplary system and method can generate multiple predictions for the same image, and then aggregate them into a final prediction. These exemplary predictions can be generated on slightly modified images, cropped with the cropping window placed in all possible locations. Doing so can be beneficial especially in images of large breasts, where only one cropping window might not cover the whole breast parenchyma. Finally, each image at the test-time can be further modified with either resizing or affine augmentation.

### Exemplary Joint Training of Object Detection and Image Classification

The exemplary AI models according to the exemplary embodiments of the present disclosure can be simultaneously trained on the two loss functions: object detection and image classification. Object detection is the task of predicting bounding-box labels, and image classification is the task of predicting image-level probabilities for the benign and malignant categories. The exemplary models can be trained to produce these two types of predictions using the breast-level labels extracted from pathology reports and bounding-box labels. Breast-level labels based on pathology reports discussed herein can be easy to get and available for all breasts which have undergone biopsies, but do not provide precise location, size or shape of lesions. Bounding-box labels discussed herein can teach models exactly where each lesion is located and what it looks like, but are laborious to collect and have been acquired only for a subset of images. Even though the bounding-box labels are only available to a small subset of exams, it provides richer and more precise training signals to the model.

In the training set of the exemplary screening mammography dataset, about 60% of the images with positive breast-level labels are missing segmentation labels. The reason is twofold. First, some lesions are mammography-occult: radiologists cannot locate these lesions on the images, even retrospectively knowing that they were present based on reports from other imaging procedures.

Second, the annotation effort is ongoing, so many images are simply not yet annotated. Utilizing these exemplary images in training the exemplary AI models for the object detection task can be problematic, since it may end up incorrectly teaching parts of the AI models that there are no lesions in those images. The AI system may choose a simple way of handling these images: these images can be used in calculating the image classification loss, but can be ignored in calculating object detection loss. This exemplary method already works well and outperforms models trained with image classification loss only.

If not done carefully, training the neural network to perform these two tasks simultaneously may hinder each other and could lead to lower performances. The exemplary system and method according to the exemplary embodiment of the present disclosure can provide various approaches the exemplary AI system and method according to the exemplary embodiment of the present disclosure has taken to prevent such potential conflicts.

### Exemplary Object Detection

Exemplary components relevant to the object detection task which worked for the AI system are described herein.

YOLOX: exemplary anchor-free architecture+SimOTA loss YOLOX does not use pre-defined anchors, which leads to simpler training and decoding phases. There are no more part-lesion anchors which used to be forced to predict negative, and this leads to reducing potentially confusing/contradictory training signals to the model since the appearance of cancer could be self-similar. YOLOX also employs an objectness head, which decouples “predicting malignancy” and “determining which prediction overlaps the most with the ground-truth label”. Instead of just matching a single box prediction with each ground-truth lesion, YOLOX can automatically determine which and how many representations to match with ground-truth labels. Further, all these label assignments are done in a globally-optimal way. This leads to a noticeable decrease in the number of false-positive predictions compared to the prior work (see Park et al., 2021).

Exemplary Generation of Pseudo-labels The missing bounding-box labels described earlier brings some imbalances in the training pipeline: the detection head is always punished for negative images, but not always encouraged to output lesions in the presence of positive images. This is because the system just ignores the detection head when positive images do not have bounding-box labels. This imbalance can become more severe in the combined screening+diagnostic dataset because most of the diagnostic dataset is not yet annotated.

It can be observed that in the combined training, using pseudo-labels can help both detection and classification performances. Using Pseudo-labels, while it might not add any new information, seems to at least help in the object imbalances. In the latest exemplary models, it even somewhat outperforms some of the models trained with screening datasets only.

### Exemplary Image Classification

Exemplary components relevant to the image classification task which worked for the exemplary AI system are described herein.

Attention-weighted average (AWA): As an exemplary alternative way to generate image-level probability predictions for each of the malignant and benign classes, exemplary methods, systems, and computer accessible medium of the present disclosure can aggregate the bounding-box predictions with the highest predicted probabilities and use their feature vectors to form an attention-weighted averaged representation, and then feed that representation to a logistic classifier as in the local module of GMIC (Shen et al., 2021). This exemplary method works well and shows robust generalization to the test set.

### Exemplary Training 2D Models on 3D Images

The exemplary systems, methods and computer-accessible medium according to the exemplary embodiments of the present disclosure can obtain a single slice from each DBT image at a time for model training. The reason for this can be as follows. First, YOLOX is 2D CNN architecture, but DBT is 3D image. Second, it is possible to utilize too much computation and GPU memory to train using the entire DBT images at a time.

The exemplary systems, methods and computer-accessible medium according to the exemplary embodiments of the present disclosure can selectively load slices for which the lesion is visible, and also prepare the bounding-box labels which are specific to the loaded slices. This is possible because there are the pixel-level segmentation labels for the entire DBT image. In other words, the exemplary system and method according to the exemplary embodiments of the present disclosure knows which slices contain the lesions, and the exact shape and sizes of the lesions in each slice. This saves the GPU memory usage and the amount of computation to make training feasible with state-of-the-art model architectures.

In addition, the exemplary systems, methods and computer-accessible medium according to the exemplary embodiments of the present disclosure can also be used to incorporate nearby slices when training DBT models. For example, instead of using a single slice as input, the exemplary system and method according to the exemplary embodiments of the present disclosure can be used to a concatenate, e.g., 3 consecutive slices as input at a time. However, this may not lead to noticeable performance improvement, and hence the exemplary system and method according to the exemplary embodiments of the present disclosure can use, e.g., single slices as input.

### Exemplary Model Architecture for Joint Object Detection and Image Classification

Partial network sharing: The exemplary vanilla network architecture, YOLOX described herein can decouple the computation of each bounding-box prediction into two components: the predicted probability of malignancy and the predicted objectness probability. The objectness probability can be the probability that the predicted bounding-box corresponds to a whole lesion. YOLOX can multiply the probability of malignancy of each bounding-box prediction with the corresponding objectness probability to compute the final probability for each bounding box.

For example, for an input image x, the backbone network outputs the hidden representation Hₓ∈ [symbol] ^{h×w×φ} where h, w, φ are hidden dimensions. YOLOX can further process the hidden representation Hx to output the probability of malignancy and the objectness probability. First, e.g., a 1×1 convolution layer with SiLU nonlinearity is applied to the hidden representation Hₓ to output another hidden representation Gₓ∈ [symbol] ^{h×w×ψ} as follows:

$${G}_{x} = {conv}_{1 \times 1} ( {H}_{x} ) . \qquad ( 1 )$$

where ψ=²⁵⁶ for YOLOX-L architecture and ψ=320 for YOLOX-X architecture. The exemplary purpose of creating this second hidden representation Gₓ can be to adjust the size of the channel dimension for the subsequent computation. Secondly, a series of convolution layers are applied to the hidden representation Gx to make predictions. The exemplary prediction of probability of malignancy Cₓ∈ [symbol] ^{h×w×l} is computed as:

$${C}_{x} = sigmoid ( {l}_{c} ( {l}_{b} ( {l}_{a} ( {G}_{x} ) ) ) ) , \qquad ( 2 )$$

where lₐ and l_{b} are parameterized as 3×3 convolution layers with SiLU nonlinearity and lc is parameterized as a 1×1 convolution layer. The objectness probabilities Oₓ∈ [symbol] ^{h×w×l} are computed as:

$${O}_{x} = sigmoid ( {l}_{f} ( {l}_{e} ( {l}_{d} ( {G}_{x} ) ) ) ) , \qquad ( 3 )$$

where l_{d} and lₑ are parameterized as 3×3 convolution layers with SiLU nonlinearity and lf is parameterized as a 1×1 convolution layer. Lastly, the final probabilities for the bounding boxes Fₓ∈ [symbol] ^{h×w×l} are computed by multiplying the probability of malignancy Cₓ and the objectness probability Oₓ as follows:

$${F}_{x} = {C}_{x} ⊙ {O}_{x} , \qquad ( 4 )$$

where ⊙ denotes an element-wise multiplication.

When utilizing breast-level labels, the exemplary system and method according to the exemplary embodiment of the present disclosure can generate image-level probability predictions ŷ_{image}∈R and then calculate binary cross-entropy loss with the breast-level label. If the image-level prediction ŷ_{image} is calculated by aggregating the bounding-box predictions Fₓ directly, then backpropagation from the image-level classification can update not only the convolution layers lₐ, l_{b}, l_{c} but also l_{d}, lₑ, l_{f}. This is suboptimal because breast-level label lacks the necessary information to train the convolution layers l_{d}, lₑ, l_{f}. Since the convolution layers l_{d}, lₑ, l_{f} must be trained with high accuracy utilizing the bounding-box labels to output the objectness probabilities, training signals from ŷ_{image} created solely from the breast-level label would likely corrupt the behaviors of these layers. This can lead to creating many false-positive bounding-box predictions, decreasing the overall performance.

Instead, the exemplary system and method according to the exemplary embodiment of the present disclosure computes the image-level probability of malignancy ŷ_{image} in a way which does not involve the convolution layers l_{d}, lₑ, l_{f}. First, the exemplary system and method according to the exemplary embodiment of the present disclosure can identify the top-K bounding-box predictions of input image x after applying non-maximum suppression to the model predictions Fₓ. Second, from the feature map l_{b}(lₐ(Gₓ)) which is an intermediate tensor in the process of creating Cₓ, the exemplary system and method according to the exemplary embodiment of the present disclosure can select a set of K vectors {\tilde{q}ₖ} which can correspond to the top-K bounding-box predictions.

Third, the exemplary system and method according to the exemplary embodiment of the present disclosure can calculate or otherwise determine the attention weights αₖ for the K feature vectors {\tilde{q}ₖ} using a gated attention mechanism (see Ilse et al., 2018) as follows:

$${α}_{k} = \frac{exp \{ {w}^{T} ( tanh ( V {\tilde{q}}_{k}^{T} ) ⊙ sigmoid ( U {\tilde{q}}_{k}^{T} ) ) \}}{{\sum }_{j = 1}^{K} exp \{ {w}^{T} ( tanh ( V {\tilde{q}}_{j}^{T} ) ⊙ sigmoid ( U {\tilde{q}}_{j}^{T} ) ) \}} , \qquad ( 5 )$$

where ⊙ denotes an element-wise multiplication, w∈ [symbol] ^{Lx1}, V∈ [symbol] ^{LxS} and U∈ [symbol] ^{LxS} are learnable parameters. For models with YOLOX-L architecture, the system sets L=64 and S=256. For the exemplary models with YOLOX-X architecture, the exemplary system and method according to the exemplary embodiment of the present disclosure can set, e.g., L=80 and S=320. Fourth, the exemplary system and method according to the exemplary embodiment of the present disclosure can generate a representation z which is an attention-weighted average of the feature vectors {\tilde{q}ₖ} as follows:

$$z = {f}_{a} ( \{ {\tilde{q}}_{k} \} ) = {\sum }_{k = 1}^{K} {α}_{k} {\tilde{q}}_{k} . \qquad ( 6 )$$

Fifth, the exemplary systems, methods and computer-accessible medium according to the exemplary embodiment of the present disclosure can apply, e.g., a fully connected layer with sigmoid nonlinearity to z to generate the image-level prediction ŷ_{image}=sigmoid(w_{image}^{T}z), where w_{image}∈ [symbol] ^{Sx1} are learnable parameters.

As a result, the exemplary image-level probability prediction ŷ_{image} can be generated by utilizing the accurate bounding-box predictions Fₓ indirectly. The training signals from the breast-level label likely do not update the convolution layers l_{d}, lₑ, l_{f}, which can preserve the performance of the bounding-box predictions Fₓ.

Selectively skipping losses: When training with images with a positive breast-level label but missing bounding-box label, the exemplary system and method according to the exemplary embodiment of the present disclosure can utilize the classification loss (e.g., calculated with image-level predictions and labels) but skip the object detection loss (e.g., calculated with bounding-box predictions and bounding-box labels). This can prevent incorrectly teaching the model that there are no lesions in breasts with biopsy-confirmed findings.

Pseudo bounding-box label generation using breast-level labels: Selectively skipping losses works well in screening datasets, but the performance slightly degrades with the combined screening+diagnostic dataset for FFDM images. The exemplary system and method according to the exemplary embodiment of the present disclosure can hypothesize that this can be because the proportion of positive images without bounding-box labels is higher in the combined dataset than in the screening dataset. To mitigate this, the exemplary systems, methods and computer-accessible medium according to the exemplary embodiment of the present disclosure can generate pseudo bounding-box labels for those positive images without bounding-box labels in the combined screening+diagnostic dataset.

The exemplary systems, methods and computer-accessible medium according to the exemplary embodiment of the present disclosure can perform inference on the aforementioned images using one of the models previously trained on screening dataset, and use resulting predictions as pseudo bounding-box labels. To improve the quality of pseudo bounding-box labels, the exemplary system and method can utilize the breast-level labels in generating pseudo bounding-box labels. For example, the exemplary system and method can obtain top-1 malignant bounding-box prediction from the images with malignant pathology and top-1 benign bounding-box prediction from the images with benign pathology as pseudo bounding-box labels. For example, the quality of pseudo bounding-box labels may not necessarily be as good as the radiologist annotation and it may not add any new information about lesions that are drastically different from the annotated ones. However, using pseudo bounding-box labels can improve the performance on combined screening+diagnostic dataset nonetheless (about 1 percentage points improvement in image-level AUC in the validation set), possibly because it mitigates the severe imbalance between positive and negative examples.

Exemplary attention-weighted average of top-K box selection: For the exemplary models with attention-weighted average of top-K box predictions, the exemplary system and method according to the exemplary embodiment of the present disclosure can be used to select top-K box per slice during slice-level training and choose top-K boxes from the entire DBT image with all slices during inference. In this exemplary manner, the exemplary system and method according to the exemplary embodiment of the present disclosure may not require loading the entire DBT image during training, yet the exemplary model can appropriate and beneficially generalize to inference on the entire DBT image by aggregating information from all slices.

Exemplary Training with DBT images when bounding-box labels are missing: Ordinarily, the bounding-box labels facilitate the sampling of slices with visible lesions during training instead of having to utilize the whole DBT image at a time. However, when a positive DBT image is not annotated, the exemplary system and method does not know which slices to load. Since loading a random slice from this image does not guarantee the presence of visible lesion on that slice, teaching the model to predict high probability of malignancy on a random slice can be detrimental. To mitigate this, instead of a random slice of DBT images, the exemplary system and method can load the corresponding C-View image. C-View images can contain most or all information from the entire breast and therefore the exemplary system and method can teach the exemplary model to predict high probability of malignancy from such positive C-View images.

### Exemplary Performance of Exemplary System

The exemplary AI models can be trained at slice-level, but DBT images are three-dimensional and involve around 70 slices on average. Described herein is how the exemplary system and method according to the exemplary embodiments of the present disclosure aggregates such slice-level predictions for each DBT image here.

Exemplary Max-Slice-Selection (MSS): exemplary systems, methods, and computer accessible medium according to the present disclosure can use an algorithm to aggregate 2D slice-level predictions for an entire 3D image while removing duplicates across different slices, which was briefly mentioned in the prior work (see Park et al., 2021) but had not been explained in full detail. To utilize the slices with the most lesion conspicuity, it is necessary to create bounding-box predictions for every single slice during inference on DBT images. However, this creates numerous duplicate predictions for the same lesion on nearby slices. To mitigate this, according to the exemplary embodiments of the present disclosure, it is possible to remove duplicates along the depth dimension before applying non-maximum suppression with the exemplary MSS system and method according to the exemplary embodiment of the present disclosure.

Since the exemplary model predicts highest probability on the slices where each suspicious lesion is most clearly visible, the exemplary MSS system and method according to the exemplary embodiment of the present disclosure captures these slices with maximum lesion conspicuity for each lesion prediction. This can be useful in numerous applications. For example, the exemplary AI system which highlights lesions on the slice with the maximum lesion conspicuity can facilitate radiologists to easily confirm the suspicious findings. In addition, it can also be useful in synthesizing AI-processed 2D images which best captures the lesion information from 3D images.

Let F_{x,i} be the probabilities of bounding-box predictions made on ith slice of image x. First, the system aggregates the box predictions F_{x,i} from all n slices and concatenates in depth dimension to create Jₓ∈ [symbol] ^{n×h×w×l}, a tensor for all bounding-box probabilities for the 3D image x. Then, the system chooses the max prediction along the depth dimension, meaning the system chooses the slice with the highest prediction at each location of the feature map (or each anchor in anchor-based networks). This creates a new tensor Mₓ∈ [symbol] ^{1×h×w×l} which contains the bounding-boxes with the highest probabilities at each xy-location of the tensor Jₓ. The assumption is that there is a single slice where the lesion is the most clearly visible (likely the center of the lesion) and that no two distinct lesions will have highly overlapping xy-coordinates. At this point, the total number of box predictions is the same as the original number of box predictions which would have been generated from a single slice of image. The system then treats these predictions as if they all come from the same slice and then apply Non-maximum suppression (NMS; duplicate removal) algorithm. Finally, the system relocates the resulting box predictions to the corresponding slices which they originated from.

This exemplary process is applied to generate a final set of bounding-box predictions for each DBT image. In addition, when performing 3D-image-level inference using AWA models, the exemplary system and method according to the exemplary embodiments of the present disclosure feeds the bounding-box predictions after this MSS algorithm.

### Exemplary Hyperparameters

The network hyperparameters were optimized with random search. The architecture was randomly chosen between YOLOX-L and YOLOX-X. The height of the input image h was randomly chosen between {1536, 1664, 1792, 1920, 2048}. The learning rate q was sampled from log-uniform distribution log_10(η)˜U(9e-07, 4e-06). The number of bounding-box predictions K used in attention-weighted average prediction was randomly chosen between {5, 6, 7, 8, 9}. The weight decay hyperparameter w was randomly sampled from log-uniform distribution log_10(ω)˜U(3e-04, 5.5e-04). The momentum p was randomly sampled from uniform distribution μ˜U(0.80, 0.92).

### Exemplary Configuration for Deployment Exemplary Updates

For an exemplary deployment, the exemplary system, method, and computer accessible medium according to the exemplary embodiment of the present disclosure imported additional segmentations (e.g., about 2,000 additional images with segmentation when counting both screening and diagnostic dataset) as well as newly updated DBT dataset (about 15,000 additional screening exams).

Training data: FFDM screening only dataset, C-View screening only dataset, DBT screening only dataset, and FFDM combined screening+diagnostic dataset.

For each training data modality, the system split the dataset 10-fold. The exemplary system and method according to the exemplary embodiment of the present disclosure took each fold as a validation set and the rest of the data as a training set. This gives, e.g., 10 different train-validation splits for each dataset.

For each of the 10 train-validation splits, the exemplary system and method according to the exemplary embodiment of the present disclosure utilized top-3 hyperparameters for each dataset according to the preliminary experiments. Therefore, the exemplary system and method according to the exemplary embodiment of the present disclosure can be training, e.g., 4*10*3=120 models. This was done to maximize the diversity between models in an ensemble.

Out of the aforementioned 120 models, a small subset can be chosen to obtain good performances efficiently using the greedy ensemble selection method (see Caruana, 2004). This is an iterative exemplary procedure that adds one model to an ensemble at a time. At each step, the model which maximizes the ensemble performance is chosen. The exemplary system further enforces an additional rule that restricts the set of candidate models at each step. Specifically, only the set of models trained on a particular dataset out of the four datasets were considered at each step, and the choice of the dataset alternated every step.

### Exemplary Additional Model Training for Existing Patients

Exemplary training methods generating models for existing patients are as follows.

Performing inference on the new examinations of the existing patients used in training the exemplary AI systems and methos can potentially risk information leak. For example, an exemplary model could have memorized the appearance of the breast for an existing patient in order to determine malignancy. When a new examination of the same patient is provided, the exemplary model can make predictions based on that memorization instead of properly assessing the breast for the sign of malignancy.

To mitigate this, the exemplary system and method according to the exemplary embodiments of the present disclosure trains additional models with held-out datasets to guarantee that there exist some models which have not seen the existing patients in either training or validation phases. For example, the dataset can be divided into, e.g., 10 subsets and use each subset as a held-out dataset. With each held-out dataset, the exemplary system and method according to the exemplary embodiments of the present disclosure trains the selected models from the greedy ensemble selection procedure with the remaining data.

### Exemplary Hyperparameters

The exemplary top-3 configuration for each modality according to the aforementioned hyperparameter search was used in the ensembles with different random seeds.

### Exemplary Experimental Results

The test AUROC of the exemplary AI models trained on different imaging modalities are provided in Table 3. The best exemplary result is achieved when ensembling the AI systems trained on all imaging modalities. The performance of the best ensemble is shown at different threshold values that lead to various percentiles in Table 4. By rejecting the exams which correspond to the bottom 45% of the model predictions, the system disclosed herein could save 32.39% of unnecessary recalls and potentially reduce radiologist workload by 45% while missing no malignancies.

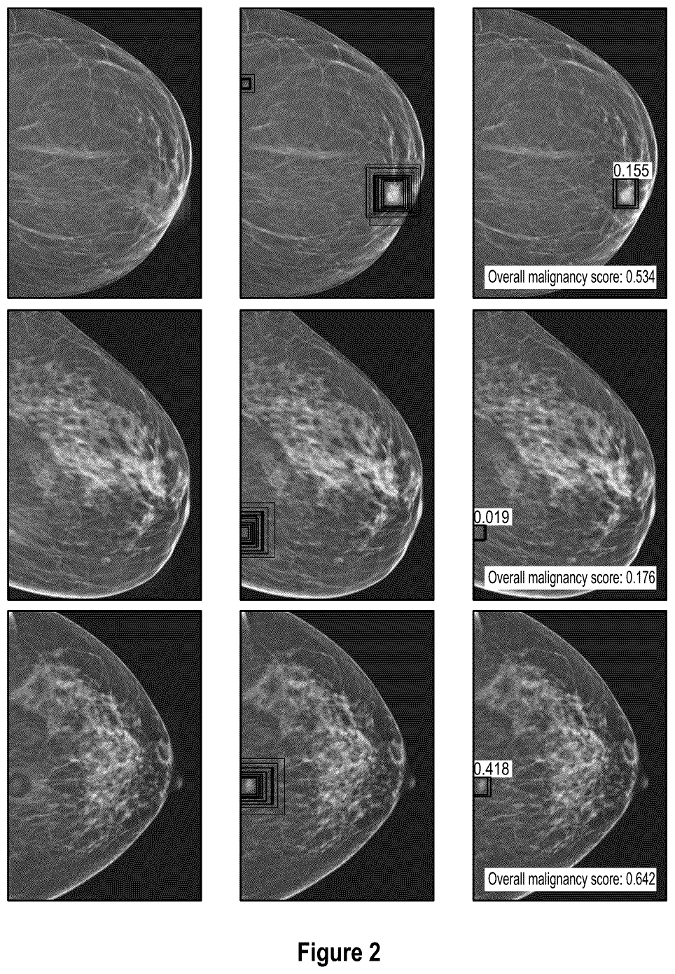

Example visualizations of malignant saliency maps and bounding-box predictions before and after duplicate removal process for exemplary validation FFDM images are shown in FIG. 2. The saliency maps are shown on the left column as red highlights over the input images. The middle column shows the bounding-box predictions before the non-maximum suppression (NMS; duplicate removal) algorithm. The right column shows the ground-truth cancer bounding-box label in red boxes, post-processed bounding-box predictions with its associated scores in green boxes, and overall image-level predictions in white box at the bottom of its image. The model is capable of accurately detecting and highlighting the cancer lesions. Compared to the prior work, the exemplary AI models according to the exemplary embodiments of the present disclosure can generate the true-positive bounding-box predictions while making less false-positive bounding-box predictions. In addition, the saliency map of the image aligns well with the biopsied lesions. However, since the test set lacks any bounding-box labels, it is not yet possible to quantify the object detection performances on the test set.

TABLE 3
Test AUROCs on the unseen 2020 Q1 data set. The best
performance is achieved by ensembling models trained
on different imaging modalities and/or datasets.

| modality | exam-level 2020 Q1 |
|---|---|
| FFDM | 0.888 |
| C-View | 0.895 |
| DBT | 0.915 |
| FFDM + C-View | 0.903 |
| C-View + DBT | 0.922 |
| FFDM + DBT | 0.927 |
| FFDM + C-View + DBT | 0.927 |

TABLE 4
The exemplary result statistics by percentile thresholds for the best ensemble.
By rejecting the exams which correspond to the bottom 45% of the model predictions,
the system disclosed herein could save 32.39% of unnecessary recalls and potentially
reduce radiologist workload by 45% while missing no malignancies.

| | | | | | | # exams | | |
|---|---|---|---|---|---|---|---|---|
| | | | | | | above | | |
| | | | | % possible | | this AI | | |
| | | | # exams | recalls that | # possible | threshold | | |
| | | # exams | rad asked | could be | recalls | that | | |
| | | with | for recall | saved based | that could | radiologist | | |
| | # exams | cancer | (BIRADS | on setting AI | be saved/ | did not | | |
| | above this | above this | 0) below | model | week | recall | | |
| percentile | AI | AI | this | at this | (enterprise- | (BIRADS | | |
| threshold | threshold | threshold | threshold | threshold | wide) | 1 or 2) | sensitivity | FN rate |
| 100 | 0 | 0 | 2470 | 100 | 190 | 0 | 0 | 1 |
| 95 | 985 | 78 | 2208 | 89.39 | 169.8 | 723 | 0.609 | 0.391 |
| 90 | 1969 | 94 | 2029 | 82.15 | 156.1 | 1528 | 0.734 | 0.266 |
| 85 | 2953 | 106 | 1854 | 75.06 | 142.6 | 2337 | 0.828 | 0.172 |
| 80 | 3937 | 112 | 1709 | 69.19 | 131.5 | 3176 | 0.875 | 0.125 |
| 75 | 4921 | 117 | 1561 | 63.20 | 120.1 | 4012 | 0.914 | 0.086 |
| 70 | 5905 | 122 | 1409 | 57.04 | 108.4 | 4844 | 0.953 | 0.047 |
| 65 | 6890 | 124 | 1280 | 51.82 | 98.5 | 5700 | 0.969 | 0.031 |
| 60 | 7874 | 124 | 1163 | 47.09 | 89.5 | 6567 | 0.969 | 0.031 |
| 55 | 8858 | 125 | 1044 | 42.27 | 80.3 | 7432 | 0.977 | 0.023 |
| 50 | 9842 | 127 | 913 | 36.96 | 70.2 | 8285 | 0.992 | 0.008 |
| 45 | 10826 | 128 | 800 | 32.39 | 61.5 | 9156 | 1 | 0 |
| 40 | 11810 | 128 | 687 | 27.81 | 52.8 | 10027 | 1 | 0 |
| 35 | 12794 | 128 | 565 | 22.87 | 43.5 | 10889 | 1 | 0 |
| 30 | 13779 | 128 | 457 | 18.50 | 35.2 | 11766 | 1 | 0 |
| 25 | 14763 | 128 | 361 | 14.62 | 27.8 | 12654 | 1 | 0 |
| 20 | 15747 | 128 | 258 | 10.45 | 19.8 | 13535 | 1 | 0 |
| 15 | 16731 | 128 | 173 | 7.00 | 13.3 | 14434 | 1 | 0 |
| 10 | 17715 | 128 | 95 | 3.85 | 7.3 | 15340 | 1 | 0 |
| 5 | 18699 | 128 | 35 | 1.42 | 2.7 | 16264 | 1 | 0 |
| 0 | 19684 | 128 | 0 | 0 | 0 | 17214 | 1 | 0 |

### Exemplary Benefits and Results of Exemplary Embodiments

1. A combination of weak+strong supervision can assist both tasks.
2. When utilizing DBT models, slice-level training and then aggregating slice-level predictions for each 3D image works reasonably well.
3. Exemplary ensembling models trained/inferenced on multiple imaging modalities reaches the best AUROC.
4. Exemplary ensembles can save, e.g., 32.39% of unnecessary recalls and potentially reduce radiologist workload by, e.g., 45% while missing no malignancies.
5. The system can also highlight the location of suspicious findings on 2D and 3D mammography images for AI decision support.

### Exemplary Diagram

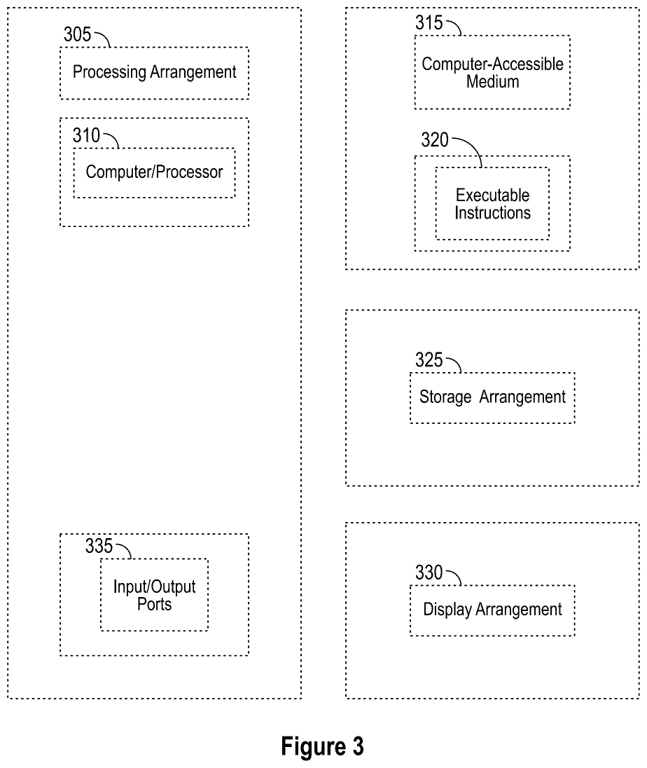

FIG. 3 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 305. Such processing/computing arrangement 305 can be, for example entirely or a part of, or include, but not limited to, a computer/processor 310 that can include, for example one or more microprocessors, and use instructions stored on a computer-accessible medium (e.g., RAM, ROM, hard drive, or other storage device).

As shown in FIG. 3, for example a computer-accessible medium 315 (e.g., as described herein above, a storage device such as a hard disk, floppy disk, memory stick, CD-ROM, RAM, ROM, etc., or a collection thereof) can be provided (e.g., in communication with the processing arrangement 305). The computer-accessible medium 315 can contain executable instructions 320 thereon. In addition or alternatively, a storage arrangement 325 can be provided separately from the computer-accessible medium 315, which can provide the instructions to the processing arrangement 305 so as to configure the processing arrangement to execute certain exemplary procedures, processes, and methods, as described herein above, for example.

Further, the exemplary processing arrangement 305 can be provided with or include an input/output ports 335, which can include, for example a wired network, a wireless network, the internet, an intranet, a data collection probe, a sensor, etc. As shown in FIG. 3, the exemplary processing arrangement 305 can be in communication with an exemplary display arrangement 330, which, according to certain exemplary embodiments of the present disclosure, can be a touch-screen configured for inputting information to the processing arrangement in addition to outputting information from the processing arrangement, for example. Further, the exemplary display arrangement 330 and/or a storage arrangement 325 can be used to display and/or store data in a user-accessible format and/or user-readable format.

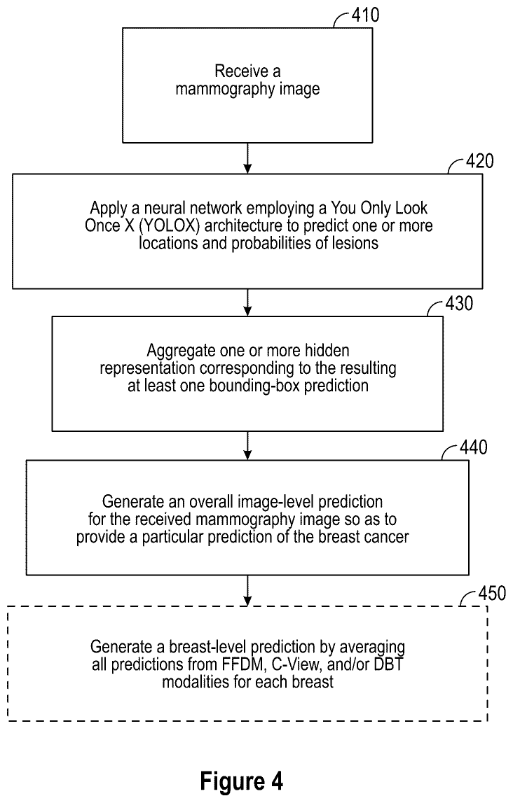

FIG. 4 shows a flow chart of a method according to an exemplary embodiment of the present disclosure. At 410, the system, method, and/or computer accessible medium may receive a mammography image. Then at 420, exemplary embodiments can apply a neural network employing a You Only Look Once X (YOLOX) architecture to predict one or more locations and probabilities of lesions. Next at 430, the exemplary system, method, and/or computer accessible medium can aggregate one or more hidden representation corresponding to the resulting at least one bounding-box prediction. Next at 440, exemplary embodiments of the present disclosure can generate an overall image-level prediction for the received mammography image so as to provide a particular prediction of the breast cancer. Finally, at 450, exemplary embodiments of the present disclosure can generate a breast-level prediction by averaging all predictions from FFDM, C-View, DBT modalities for each breast.

According to exemplary embodiments of the present disclosure, numerous specific details have been set forth. It is to be understood, however, that implementations of the disclosed technology can be practiced without these specific details. In other instances, well-known methods, structures and techniques have not been shown in detail in order not to obscure an understanding of this description. References to “some examples,” “other examples,” “one example,” “an example,” “various examples,” “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrases “in one example,” “in one exemplary embodiment,” or “in one implementation” does not necessarily refer to the same example, exemplary embodiment, or implementation, although it may.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification and drawings, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

Throughout the disclosure, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form. This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

### EXEMPLARY REFERENCES

The following references are hereby incorporated by references, in their entireties:
1. Hildegunn S Aase, Åsne S Holen, Kristin Pedersen, Nehmat Houssami, Ingfrid S Haldorsen, Sofie Sebuødegård, Berit Hanestad, and Solveig Hofvind. A randomized controlled trial of digital breast tomosynthesis versus digital mammography in population-based screening in bergen: interim analysis of performance indicators from the to-be trial. European radiology, 29(3):1175-1186, 2019.
2. James Bergstra, and Yoshua Bengio. “Random search for hyper-parameter optimization.” Journal of machine learning research, 13.2, 2012.
3. Stephen W Duffy, Laszlo Tabir, Hsiu-Hsi Chen, Marit Holmqvist, Ming-Fang Yen, Shahim Abd-salah, Birgitta Epstein, Ewa Frodis, Eva Ljungberg, Christina Hedborg-Melander, et al. The impact of organized mammography service screening on breast carcinoma mortality in seven swedish counties: a collaborative evaluation. Cancer: Interdisciplinary International Journal of the American Cancer Society, 95(3):458-469, 2002.
4. Joann G Elmore, Gary M Longton, Patricia A Carney, Berta M Geller, Tracy Onega, Anna N A Tosteson, Heidi D Nelson, Margaret S Pepe, Kimberly H Allison, Stuart J Schnitt, et al. Diagnostic concordance among pathologists interpreting breast biopsy specimens. JAMA, 313(11): 1122-1132, 2015.
5. Shijie Fang, Yuhang Cao, Xinjiang Wang, Kai Chen, Dahua Lin, and Wayne Zhang. Wssod: A new pipeline for weakly- and semi-supervised object detection. arXiv preprint arXiv: 2105.11293, 2021.
6. Zheng Ge, Songtao Liu, Feng Wang, Zeming Li, and Jian Sun. Yolox: Exceeding yolo series in 2021. arXiv: 2107.08430, 2021.
7. Judith M Hemmer, Johannes C Kelder, and Hans P M van Heesewijk. Stereotactic large-core needle breast biopsy: analysis of pain and discomfort related to the biopsy procedure. European Radiology, 18(2):351-354, 2008.
8. Joao V Horvat, Delia M Keating, Halio Rodrigues-Duarte, Elizabeth A Morris, and Victoria L Mango. Calcifications at digital breast tomosynthesis: imaging features and biopsy techniques. Radiographics, 39(2):307, 2019.
9. N Howlader, A M Noone, M Krapcho, D Miller, A Brest, M Yu, J Ruhl, Z Tatalovich, A Mariotto, DR Lewis, et al. Seer cancer statistics review, 1975-2017. National Cancer Institute, 2020.
10. Kathryn L Humphrey, Janie M Lee, Karen Donelan, Chung Y Kong, Olubunmi Williams, Omosalewa Itauma, Elkan F Halpern, Beverly J Gerade, Elizabeth A Rafferty, and J Shannon Swan. Percutaneous breast biopsy: effect on short-term quality of life. Radiology, 270(2):362-368, 2014.
11. Maximilian Ilse, Jakub M Tomczak, and Max Welling. Attention-based deep multiple instance learning. arXiv: 1802.04712, 2018.
12. Daniel B Kopans. Beyond randomized controlled trials: organized mammographic screening substantially reduces breast carcinoma mortality. Cancer, 94(2):580-581, 2002.
13. Daniel B Kopans. An open letter to panels that are deciding guidelines for breast cancer screening.
14. Breast Cancer Research and Treatment, 151(1):19-25, 2015.
15. James R Maxwell, Mary E Bugbee, David Wellisch, Anat Shalmon, James Sayre, and Lawrence W Bassett. Imaging-guided core needle biopsy of the breast: study of psychological outcomes. The Breast Journal, 6(1):53-61, 2000.
16. Jungkyu Park, Yoel Shoshan, Robert Marti, Pablo Gómez del Campo, Vadim Ratner, Daniel Khapun, Aviad Zlotnick, Ella Barkan, Flora Gilboa-Solomon, Jakub Chledowski, Jan Witowski, Alexandra Millet, Eric Kim, Alana Lewin, Kristine Pysarenko, Sardius Chen, Julia Goldberg, Shalin Patel, Anastasia Plaunova, Melanie Wegener, Stacey Wolfson, Jiyon Lee, Sana Hava, Sindhoora Murthy, Linda Du, Sushma Gaddam, Ujas Parikh, Laura Heacock, Linda Moy, Beatriu Reig, Michal Rosen-Zvi, and Krzsyztof J Geras. Lessons from the first dbtex challenge. Nature Ma-chine Intelligence, 3(8):735-736, 2021.
17. Yiqiu Shen, Nan Wu, Jason Phang, Jungkyu Park, Kangning Liu, Sudarshini Tyagi, Laura Heacock, Gene Kim, Linda Moy, Kyunghyun Cho, and Krzysztof J Geras. An interpretable classifier for high-resolution breast cancer screening images utilizing weakly supervised localization. Medical Image Analysis, 68:101908, 2021.
18. Melvin Silverstein. Where's the outrage?Journal of the American College of Surgeons, 208(1): 78-79, 2009.
19. Melvin J Silverstein, Abram Recht, Michael D Lagios, Ira J Bleiweiss, Peter W Blumencranz, Terri Gizienski, Steven E Harms, Jay Harness, Roger J Jackman, V Suzanne Klimberg, et al. Special report: Consensus conference iii. image-detected breast cancer: state-of-the-art diagnosis and treatment. Journal of the American College of Surgeons, 209(4):504-520, 2009.
20. Hyuna Sung, Jacques Ferlay, Rebecca L Siegel, Mathieu Laversanne, Isabelle Soerjomataram, Ahmedin Jemal, and Freddie Bray. Global cancer statistics 2020: Globocan estimates of incidence and mortality worldwide for 36 cancers in 185 countries. CA: a cancer journalfor clinicians, 71 (3):209-249, 2021.
21. Mingxing Tan, Ruoming Pang, and Quoc V Le. Efficientdet: Scalable and efficient object detection. In Proceedings of the IEEE/CVF Conference on Computer Vision and Pattern Recognition, pp. 10781-10790, 2020.
22. U.S. Food and Drug Administration. MQSA national statistics, 2021. URL https://www.fda.gov/radiation-emitting-products/mqsa-insights/mqsa-national-statistics.
23. Anna Vlahiotis, Brian Griffin, A Thomas Stavros, and Jay Margolis. Analysis of utilization patterns and associated costs of the breast imaging and diagnostic procedures after screening mammography. ClinicoEconomics and Outcomes Research: CEOR, 10:157, 2018.
24. Nan Wu, Jason Phang, Jungkyu Park, Yiqiu Shen, S Gene Kim, Laura Heacock, Linda Moy, Kyunghyun Cho, and Krzysztof J Geras. The NYU breast cancer screening dataset v1. New York Univ., New York, NY, USA, Tech. Rep, 2019.
25. Paul A. Yushkevich, Joseph Piven, Heather Cody Hazlett, Rachel Gimpel Smith, Sean Ho, James C. Gee, and Guido Gerig. User-guided 3D active contour segmentation of anatomical structures: Significantly improved efficiency and reliability. Neuroimage, 31(3):1116-1128, 2006.
26. Rich Caruana, Alexandru Niculescu-Mizil, Geoff Crew, Alex Ksikes. Ensemble selection from libraries of models. In Proceedings of the twenty-first international conference on Machine learning, p. 18, 2004.

1. An artificial intelligence (AI) system for detecting a breast cancer using mammography, comprising:
at least one computer processor configured to perform procedures comprising:
receiving a mammography image;
applying a neural network employing a You Only Look Once X (YOLOX) architecture to predict one or more locations and probabilities of lesions; and
generating an overall image-level prediction for the received mammography image so as to provide a particular prediction of the breast cancer.

2. The AI system of claim 1, wherein the at least one computer processor is further configured to aggregate one or more hidden representation corresponding to the resulting at least one bounding-box prediction.

3. The AI system of claim 1, wherein the mammography image is at least one of a digital breast tomosynthesis (DBT) 3D image, a full-field digital mammography (FFDM) image, or a C-View image.

4. The AI system of claim 2, wherein the at least one computer processor is further configured to generate a breast-level prediction by averaging a plurality of image level predictions from FFDM, C-View, DBT modalities for each breast

5. The AI system of claim 1, wherein the mammography image is a digital breast tomosynthesis (DBT) 3D image, and wherein the at least one computer processor is further configured to performed a maximum intensity projection along a depth axis to match dimensions of a corresponding C-View image associated with the mammography image.

6. The AI system of claim 1, wherein the overall image-level prediction is separately performed on each 2D slice in the DBT 3D image, and wherein the at least one computer processor is further configured to generate a final 3D-image-level prediction for the DBT 3D image by aggregating the predictions and one or more corresponding feature vectors for individual 2D slices associated with the mammography image using a Max-Slice-Selection (MSS) procedure.

7. The AI system of claim 1, wherein each of the at least one bounding box predictions is defined by a plurality of segmentation components defining the contours of a predicted lesion.

8. The AI system of claim 1, wherein the YOLOX architecture is a YOLOX-1 architecture or a YOLOX-x architecture.

9. The AI system of claim 1, wherein the at least one computer processor is further configured to utilize the mammography image to produce an image-level probability of malignancy using breast-level labels extracted from bounding-box labels.

10. The AI system of claim 1, wherein the at least one computer processor is further configured to generate at least one bounding-box prediction of one or more locations and probabilities of lesions based on the mammography image and wherein the at least one image-wise prediction is generated based on the at least one bounding-box prediction.

11. An artificial intelligence (AI) method for detecting a breast cancer using mammography, comprising:
receiving a mammography image;
applying a neural network employing a You Only Look Once X (YOLOX) architecture to predict one or more locations and probabilities of lesions; and
generating an overall image-level prediction for the received mammography image so as to provide a particular prediction of the breast cancer.

12. The AI method of claim 11, further comprising, aggregating one or more hidden representation corresponding to the resulting at least one bounding-box prediction.

13. The AI method of claim 11, wherein the mammography image is at least one of a digital breast tomosynthesis (DBT) 3D image, a full-field digital mammography (FFDM) image, or a C-View image.

14. The AI system of claim 13, further comprising, generating a breast-level prediction by averaging a plurality of image level predictions from FFDM, C-View, DBT modalities for each breast.

15. The AI method of claim 11, wherein the overall image-level prediction is separately performed on each 2D slice in the DBT 3D image, and wherein a final 3D-image-level prediction is generated for the DBT 3D image by aggregating the predictions and one or more corresponding feature vectors for individual 2D slices associated with the mammography image using a Max-Slice-Selection (MSS) procedure.

16. The AI method of claim 11, wherein each of the at least one bounding box predictions is defined by a plurality of segmentation components defining the contours of a predicted lesion.

17. The AI method of claim 11, wherein the YOLOX architecture is a YOLOX-1 architecture or a YOLOX-x architecture.

18. The AI method of claim 11, further comprising, utilizing the mammography image to produce an image-level probability of malignancy using breast-level labels extracted from bounding-box labels.

19. The AI method of claim 11, further comprising, generating at least one bounding-box prediction of one or more locations and probabilities of lesions based on the mammography image and wherein the at least one image-wise prediction is generated based on the at least one bounding-box prediction.

20. A non-transitory, computer-readable medium for detecting a breast cancer using mammography comprising instructions that, when executed on a computer artificial intelligence (AI) system, cause the computer system to perform procedures comprising:
receiving a mammography image;
applying a neural network employing a You Only Look Once X (YOLOX) architecture to predict one or more locations and probabilities of lesions; and
generating an overall image-level prediction for the received mammography image so as to provide a particular prediction of the breast cancer.

21-26. (canceled)
